(19) United States
(12) Patent Application Publication
(10) Pub. No.: US 2009/0095681 A1
(43) Pub. Date: Apr. 16, 2009

(54) P-TERT-BUTYLCALIX[6]ARENES WITH TRIACIDIC FUNCTIONS AT 2, 4 AND 6, SUPPORTED LIQUID MEMBRANES AND SUPPORT MATERIALS COMPRISING THE SAME AND USE THEREOF

(75) Inventors: Raphael Duval, Deauville (FR); Catherine Cossonet, Igny (FR); Celine Bouvier-Capely, Chatillon (FR)

Correspondence Address: YOUNG & THOMPSON, 209 Madison Street, Suite 500, ALEXANDRIA, VA, 22314, US

(73) Assignees: CHELATOR, Deauville (FR); INSTITUT DE RADIOPROTECTION ET DE SURETE NUCLEAIR (I.R.S.N.), FONTENAY AUX ROSES (FR)

(21) Appl. No.: 11/914,861

(22) PCT Filed: May 16, 2006
(86) PCT No.: PCT/FR2006/001105

§ 371 Date: Nov. 12, 2008

(30) Foreign Application Priority Data
May 17, 2005 (FR) 0504944

(51) Int. Cl. B01D 15/08; C07C 59/11; C07C 237/14; C08F 12/08; C08F 20/56; C08F 14/00; B32B 5/16; G01N 33/20

(52) U.S. Cl. 210/656; 562/480; 564/153; 436/74; 210/198.2; 428/402; 525/333.6; 525/330.7; 525/329.4; 525/330.5

(57) ABSTRACT

The invention relates to novel p-tert butylcalix[6]arenes of formulae (IA) and (IB) with carboxylic or hydroxyamino triacidic functions in positions 2, 4 and 6, and other functions in positions 1, 3 and 5, supported liquid membranes and support materials comprising the above and the uses thereof.

During the fuel cycle, from the uranium mine to the reprocessing plants, the radioactive compounds are present in highly varied physicochemical forms generating different toxicities and thus bringing about highly diversified risks of exposure to the personnel.

In order to ensure protection of the health of the workers, it is necessary to carry out medical monitoring. Different examinations are carried out on the workers, including the analysis of the alpha-emitting actinides eliminated in the excreta (urine and stools).

Currently, the analytical technique of reference is the α spectrometry. Due to the short distance covered by a particles in the material, it is impossible to directly measure uranium, americium and plutonium in urine; it is thus necessary to manufacture a thin film source for each of the actinides. This involves a first stage of mineralization of the sample, followed by a chemical purification which makes it possible to isolate the actinides from the urinary matrix and to separate them from one another, in order to limit spectral interferences.

In the protocols currently used by all laboratories for radiotoxicological analysis, this purification is based on successive separations of the actinides using appropriate chromatographic columns (Harduin et al., Radioprotection, 31, No. 2, 229-245, 1996). These protocols are lengthy (6 days in order to obtain the final result), in the order of 3 days for the chemical treatment and 3 days of counting by α spectrometry, in order to achieve levels of activity per isotrope of less than 1 mBq.l⁻¹ of urine.

In the context of continual technological change in the nuclear industry and of change in regulations, the means which make it possible to routinely monitor the workers and to evaluate the internal exposure of individuals in the event of accidents must be improved.

There is thus a real and crucial need in terms of health to be able to have available, within very short periods of time, results of measurements relating to uranium, americium and plutonium in order to be able to counter the deleterious effects of alpha radiation.

Making available faster and more efficient analytical techniques would also make it possible to monitor α emitters in the environment, in order to respond to the problems of long-term development.

Numerous studies have been carried out in order to solve this problem but none has been successful to date. Research has been directed at specific complexing agents for the three actinides, in order to extract them from complex matrices, such as biological media.

The majority of the studies have been carried out on specific complexing cages: the calixarenes. For example, in 1993, Araki et al. showed the complexing properties in liquid/liquid extraction of 1,3,5-O-trimethyl-2,4,6-O-tri(carboxylic acid)-para-tert-butylcalix[6]arene (molecule represented hereinafter by the formula IA) with regard to uranium (Chem. Lett., 829-832, 1993). In 1994, Van Duynoven et al. used the same molecule to study its conformational equilibria (J. Am. Chem. Soc., 116, 5814-5822). In 1997, C. Dise et al. (Radioprotection, 32, No. 5, 659-671) demonstrated the selectivity, in liquid/liquid extraction, of the molecule of formula A with regard to uranium in the presence of plutonium and sodium.

More recently, a novel extraction agent, a synthetic derivative of the molecule of formula A, 1,3,5-O-trimethyl-2,4,6-O-tri(hydroxamic acid)-para-tert-butylcalix[6]arene (molecule represented hereinafter by the formula B), was proposed by Bennoura et al. in Journal of Inclusion Phenomena and Macrocyclic Chemistry, 40, 95-98, 2001, for the complexing of cations.

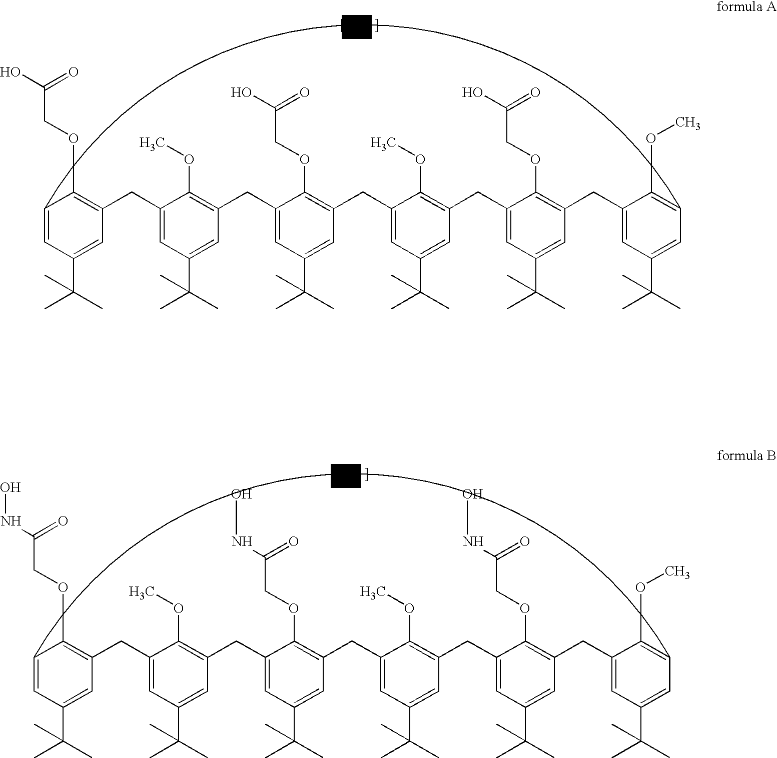

To our knowledge, the molecules of formulae A and B have never formed the subject of studies on supported membranes or on grafted supports.

The immobilization of calixarenes via a covalent bond on support materials has already been described by S. P. Alexandratos et al. in Macromolecules, 2001, 34, 206-210, and then in 2002 by Trivedi et al. in Reactive and Functional Polymers, 50, 205-216. Furthermore, the work by Z. Asfari, published in 2001 by Kluwer Academic Press (Netherlands) and entitled “Calixarenes”, presents, in the chapter written by R. Milbradt and V. Böhmer, pages 663 to 676, a review of the immobilization techniques and the products obtained. However, the immobilization of calixarenes of formulae A and B has never been described.

None of the products or techniques described in the prior art makes it possible to analyze uranium and/or americium and/or plutonium cations at a content in the order of 1 mBq per liter in less than 6 days.

Surprisingly, the inventors have found that the replacement of a methoxy group in the formulae A and B described in the literature by another group, and more advantageously by the hydroxyl group, did not reduce the complexing capabilities with regard to the uranium, americium and plutonium cations.

This discovery has been taken advantage of in preparing supported liquid membranes comprising the compounds of general formulae IA and/or IB and novel support materials. These membranes and these support materials exhibit the property of selectively complexing uranium and/or americium and/or plutonium. The use of these supported membranes in columns suitable for the analysis by extraction chromatography makes it possible for the analysis of the abovementioned elements at a content in the order of 1 mBq/l.

Thus, the present invention relates to a novel family of para-tert-butylcalix[6]arene compounds of formula (IA) or (IB)

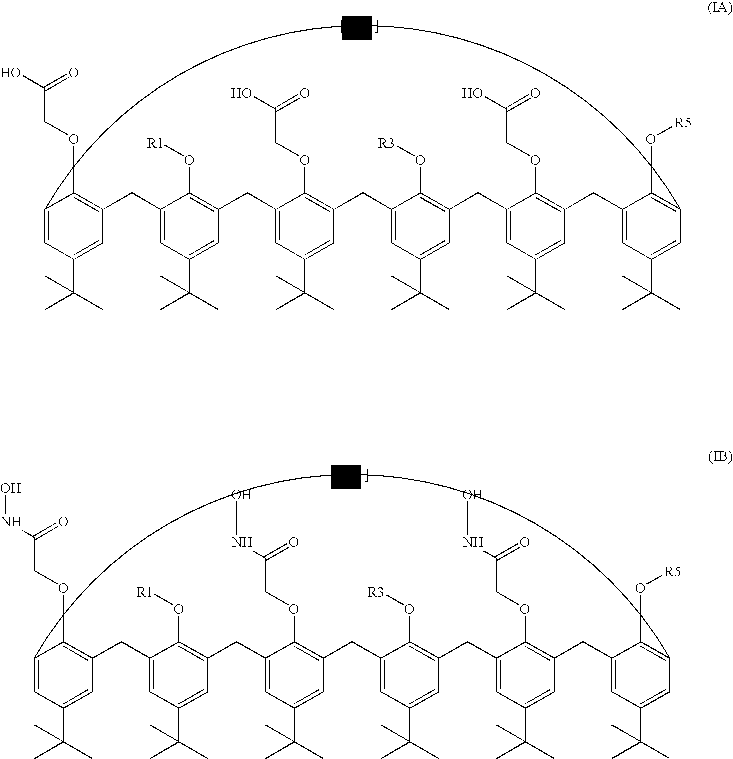

where R1, R3 and R5, which are identical or different, represent, each independently:
(i) a hydrogen or halogen atom,
(ii) an acetyl, amino, phosphate, nitro, sulfate, carboxyl, carboxylic, thiocarboxyl, carbamate or thiocarbamate radical,
(iii) an optionally substituted linear or branched alkyl having 1 to 60, preferably 1 to 30, carbon atoms which optionally exhibits at least one ethylenic or acetylenic unsaturation,
(iv) an optionally substituted cycloalkyl having from 3 to 12 carbon atoms which optionally exhibits at least one ethylenic or acetylenic unsaturation,
(v) an optionally substituted aryl, an optionally substituted naphthyl, an optionally substituted aryl(C₁-C₃₀ alkyl) or an optionally substituted (C₁-C₃₀ alkyl)aryl;
   it being possible for the radicals (ii) to (v) to be substituted by halogen atoms, organometallic compounds, alcohol, amine, carboxylic, sulfonic, sulfuric, phosphoric, phosphonic or hydroxamic acid or ester, carbamate, thiocarbamate, ether, thiol, epoxide, thioepoxide, isocyanate or isothiocyanate functional groups or it being possible for a carbon of these radicals to be replaced by a nitrogen, sulfur, phosphorus, oxygen, boron or arsenic heteroatom;
(vi) a polymer chosen from the group comprising of polystyrenes, copolymers of chloro- and/or bromomethylstyrene and of divinylbenzene, polyethers, polyacrylamides, poly(glycidyl methacrylate)s, dextrans and agaroses;
   with the following conditions:
   R1, R3 and R5 not simultaneously representing CH₃ in (IA) and (IB),
   R1, R3 and R5 not simultaneously representing CH₂COOH in (IA), and
   R1, R3 and R5 not simultaneously representing CH₂CONHOH in (IB).

According to an advantageous embodiment, in formula (IA) and (IB), two from R1, R3 and R5 represent hydrogen or methyl, the third being chosen from (vi) a polymer chosen from the group comprising of polystyrenes, copolymers of chloro- and/or bromomethylstyrene and of divinylbenzene, polyethers, polyacrylamides, poly(glycidyl methacrylate)s, dextrans and agaroses.

According to another advantageous embodiment, the para-tert-butylcalix[6]arenes of the invention are compounds of formula (IA) or (IB) in which R1, R3 and R5 are identical and preferably represent a hydrogen.

The invention also relates to supported liquid membranes comprising a para-tert-butylcalix[6]arene of formula (IA) or (IB)

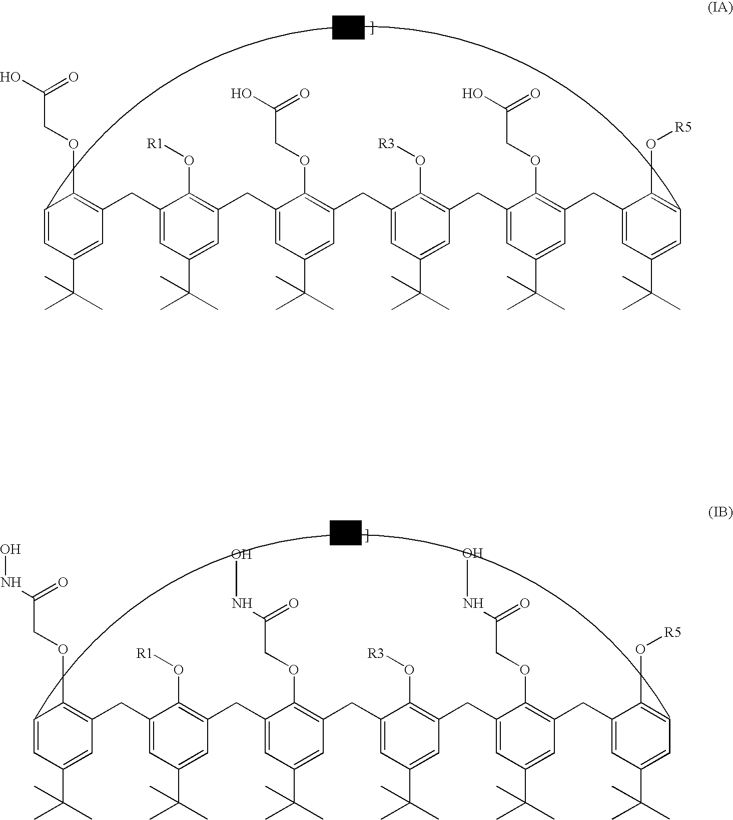

where R1, R3 and R5, which are identical or different, represent, each independently:
(i) a hydrogen or halogen atom,
(ii) an acetyl, amino, phosphate, nitro, sulfate, carboxyl, carboxylic, thiocarboxyl, carbamate or thiocarbamate radical,
(iii) an optionally substituted linear or branched alkyl having 1 to 60, preferably 1 to 30, carbon atoms which optionally exhibits at least one ethylenic or acetylenic unsaturation,
(iv) an optionally substituted cycloalkyl having from 3 to 12 carbon atoms which optionally exhibits at least one ethylenic or acetylenic unsaturation,
(v) an optionally substituted aryl, an optionally substituted naphthyl, an optionally substituted aryl(C₁-C₃₀ alkyl) or an optionally substituted (C₁-C₃₀ alkyl)aryl;
   it being possible for the radicals (ii) to (v) to be substituted by halogen atoms, organometalilic compounds, alcohol, amine, carboxylic, sulfonic, sulfuric, phosphoric, phosphonic or hydroxamic acid or ester, carbamate, thiocarbamate, ether, thiol, epoxide, thioepoxide, isocyanate or isothiocyanate functional groups or it being possible for a carbon of these radicals to be replaced by a nitrogen, sulfur, phosphorus, oxygen, boron or arsenic heteroatom;
(vi) a polymer chosen from the group comprising of polystyrenes, copolymers of chloro- and/or bromomethylstyrene and of divinylbenzene, polyethers, polyacrylamides, poly(glycidyl methacrylate)s, dextrans and agaroses;
   said product of formula (IA) or (IB) being dissolved in an organic solvent and absorbed on a support.

The organic solvent according to the invention exhibits a boiling point of greater than 60° C., so as to limit the evaporation of said solvent during storage. Furthermore, it must have good solubility properties with regard to the calixarenes of the invention. The following will be noted among the most advantageous solvents, without, however, this list being limiting: toluene, xylene, chlorobenzene, ortho-dichlorobenzene, nitrobenzene, 1,4-diisopropylbenzene, hexylbenzene, kerosene, tetrahydropyran, 1,2,3,4-tetrahydro-naphthalene, pentanol and higher homologous alcohols, glycols and their ethers, such as, for example, diethylene glycol dibutyl ether, esters, such as methyl benzoate, or ethers, such as ortho-nitrophenyl pentyl ether or nitrophenyl octyl ether.

By way of example, the solubilities in mol/l of the compound IA in various solvents are as follows: at 25° C. 1,2-dichlorobenzene: 3.13×10⁻³ M, chlorobutane: 1.6×10⁻³ M, isooctane: 1.25×10⁻³ M, isobutyl acetate: 1.75×10⁻³ M, tert-butyl acetate: 5.25×10⁻³ M, isoamyl benzoate: 2.40×10⁻³ M, benzylacetate: 3.71×10⁻³ M, methyl benzoate: 6.41×10⁻³ M, benzonitrile: 1.81×10⁻³ M, 1-hexanol: 17.65×10⁻³ M, 1-heptanol: 14.28×10⁻³ M, diethylene glycol dimethyl ether: 23.53×10⁻³ M, diethylene glycol tert-butyl ethyl ether: 11.34×10⁻³ M, diethylene glycol dibutyl ether: 19.33×10⁻³ M, dipentyl ether: 2.32×10⁻³ M, isoamyl ether: 2.02×10⁻³ M, isobutyl ether: 1.58×10⁻³ M, 1,1,2-trichlorotri-fluoroethane: 1.57×10⁻³ M, 1,2,3,4-tetrahydro-naphthalene: 6.4×10⁻³ M.

The constituent support materials of the supported liquid membranes of the invention are of inorganic origin, chosen from the group comprising of silica gels, oxides, such as alumina, zirconia and titanium oxide, or of organic origin, chosen from the group comprising of polystyrene/divinylbenzenes, polyethers, polyacrylamides and poly(glycidyl methacrylate)s, or of organo-inorganic origin, chosen from the group comprising of silica/dextran and hydroxyapatite/agarose composites, and their mixtures.

Preferably, the support is in the form of particulates, the particle size varying between 10 nm and 10 mm, preferably between 10 and 50 microns, and the porediameter varying between 10 and 5000 Å, preferably between 100 and 500 Å.

The invention also relates to the liquid membranes present in the supported liquid membranes, that is to say to the para-tert-butylcalix[6]arenes of formulae (IA) and (IB), in solution in a solvent, the solvent being a water-insoluble organic solvent as described above.

The invention also relates to a support material which is a para-tert-butylcalix[6]arene of formula (IIA) or (IIB)

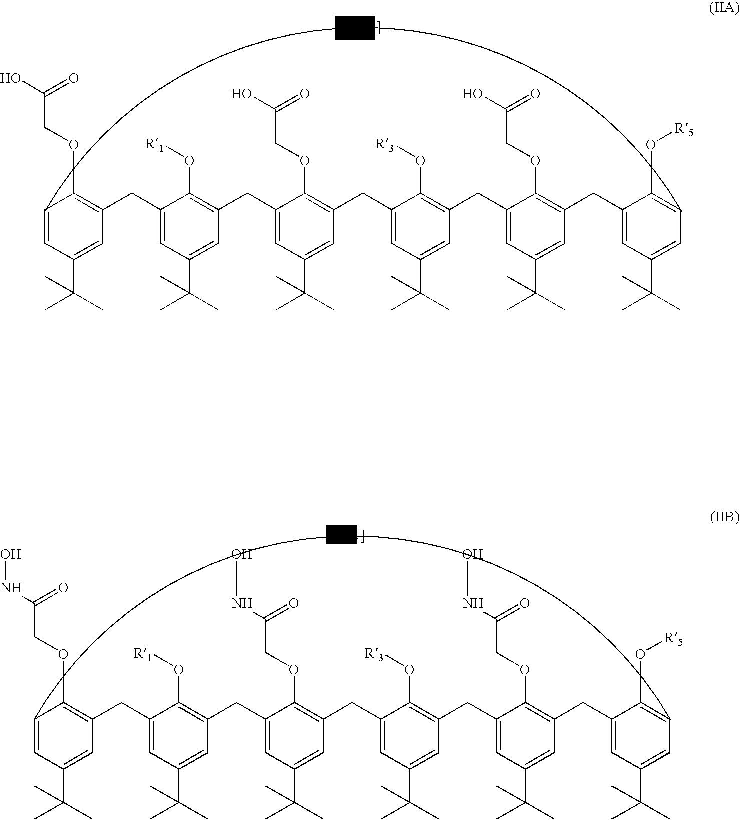

where R′1, R′3 and R′5, which are identical or different, represent, each independently:
(i) a hydrogen or halogen atom,
(ii) an acetyl, amino, phosphate, nitro, sulfate, carboxyl, carboxylic, thiocarboxyl, carbamate or thiocarbamate radical,
(iii) an optionally substituted linear or branched alkyl having 1 to 60, preferably 1 to 30, carbon atoms which optionally exhibits at least one ethylenic or acetylenic unsaturation,
(iv) an optionally substituted cycloalkyl having from 3 to 12 carbon atoms which optionally exhibits at least one ethylenic or acetylenic unsaturation,
(v) an optionally substituted aryl, an optionally substituted naphthyl, an optionally substituted aryl(C₁-C₃₀ alkyl) or an optionally substituted (C₁-C₃₀ alkyl)aryl;
   it being possible for the radicals (ii) to (v) to be substituted by halogen atoms, organometallic compounds, alcohol, amine, carboxylic, sulfonic, sulfuric, phosphoric, phosphonic or hydroxamic acid or ester, carbamate, thiocarbamate, ether, thiol, epoxide, thioepoxide, isocyanate or isothiocyanate functional groups or it being possible for a carbon of these radicals to be replaced by a nitrogen, sulfur, phosphorus, oxygen, boron or arsenic heteroatom;
(vi) a polymer chosen from the group comprising of polystyrenes, copolymers of chloro- and/or bromomethylstyrene and of divinylbenzene, polyethers, polyacrylamides, poly(glycidyl methacrylate)s, dextrans and agaroses;
(vii) -SPACER-SUPPORT,
   the SPACER being a divalent radical chosen from the group comprising of C₁-C₆₀, preferably C₁-C₃₀, alkylenes, (C₁-C₆₀ alkyl)arylenes, aryl(C₁-C₆₀ alkylenes) and aryl(C₁-C₆₀ alkyl)aryls, it being possible for the divalent radical to be substituted by halogen atoms, organometallic compounds, alcohol, amine, acid, ester, carbamate, thiocarbamate, ether, thiol, epoxide, thioepoxide, isocyanate or isothiocyanate functional groups or it being possible for a carbon of this divalent radical to be replaced by a nitrogen, sulfur, phosphorus, oxygen, boron or arsenic heteroatom; the SUPPORT being chosen from supports of inorganic or organic or organo-inorganic origin, preferably particulate supports, the particle size of which varies between 10 nm and 10 mm, preferably between 10 and 50 microns, and the pore diameter of which varies between 10 and 5000 Å, preferably between 100 and 500 Å;
   provided that at least one of R′1, R′3 or R′5 is a (vi) or (vii) group.

A support material is constituent of the stationary phase of a chromatographic column.

In the support material of the invention, the SUPPORT is of inorganic origin, chosen from the group comprising of a silica gel, oxides, such as alumina, zirconia and titanium oxide, or of organic origin, chosen from the group comprising of polystyrene/di-vinylbenzenes, polyethers, polyacrylamides and poly(glycidyl methacrylate)s, or of organo-inorganic origin, chosen from the group comprising of silica/dextran or hydroxyapatite/agarose composites.

This SUPPORT results from a support comprising reactive chemical functional groups, it being possible for said chemical functional groups to be organic or inorganic, such as, for example, carboxylic acid chloride, amine, aldehyde, thiol, sulfonyl chloride, isocyanate or metal halide functional groups, without, however, this list being limiting.

By way of example, functionalized particulate supports are the copolymer of [5-(4-(chloromethyl)phenyl)pentyl]styrene and of divinylbenzene and also the copolymer of [5-(4-(bromomethyl)phenyl)pentyl]styrene and of divinylbenzene, and the copolymer of 4-(chloromethyl)styrene and of divinylbenzene.

They can be provided in the form of spherical or irregular solid particles, the particle size of which can vary, and also the porosity. Various commercial grades are available, such as the Stratosphere® resins sold by Aldrich.

The following procedure is used to synthesize the novel compounds of general formula IA and IB:

a compound 1,3,5-trimethoxy-para-tert-butylcalix[6]-arene of formula (a)

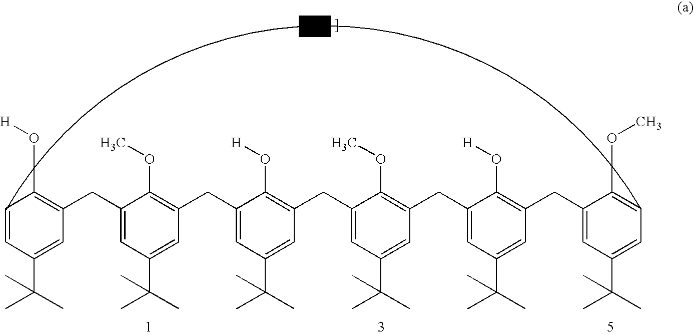

is synthesized and then a precursor compound 2,4,6-tri(ethyl ester)-1,3,5-trimethoxy-para-tert-butylcalix-[6]arene of formula (b)

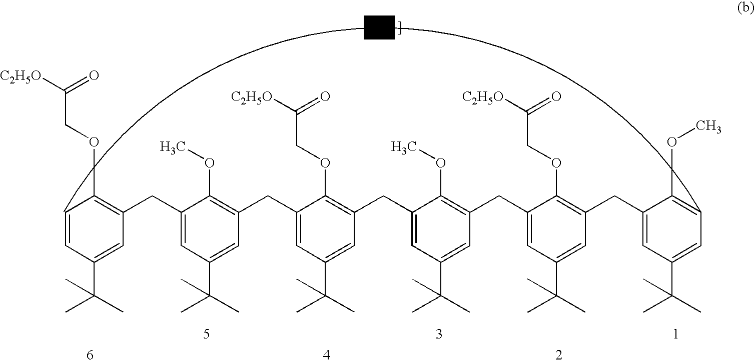

is synthesized.

The compound of formula (b) is then optionally subsequently modified, by saponification or by substitution of the carboxylic acid ethyl ester functional groups, to result in the compounds of formula A or B.

The compound of formula (b) can also be partially or completely demethylated, to result in the compounds of general formula (c1), (c2) or (c3).

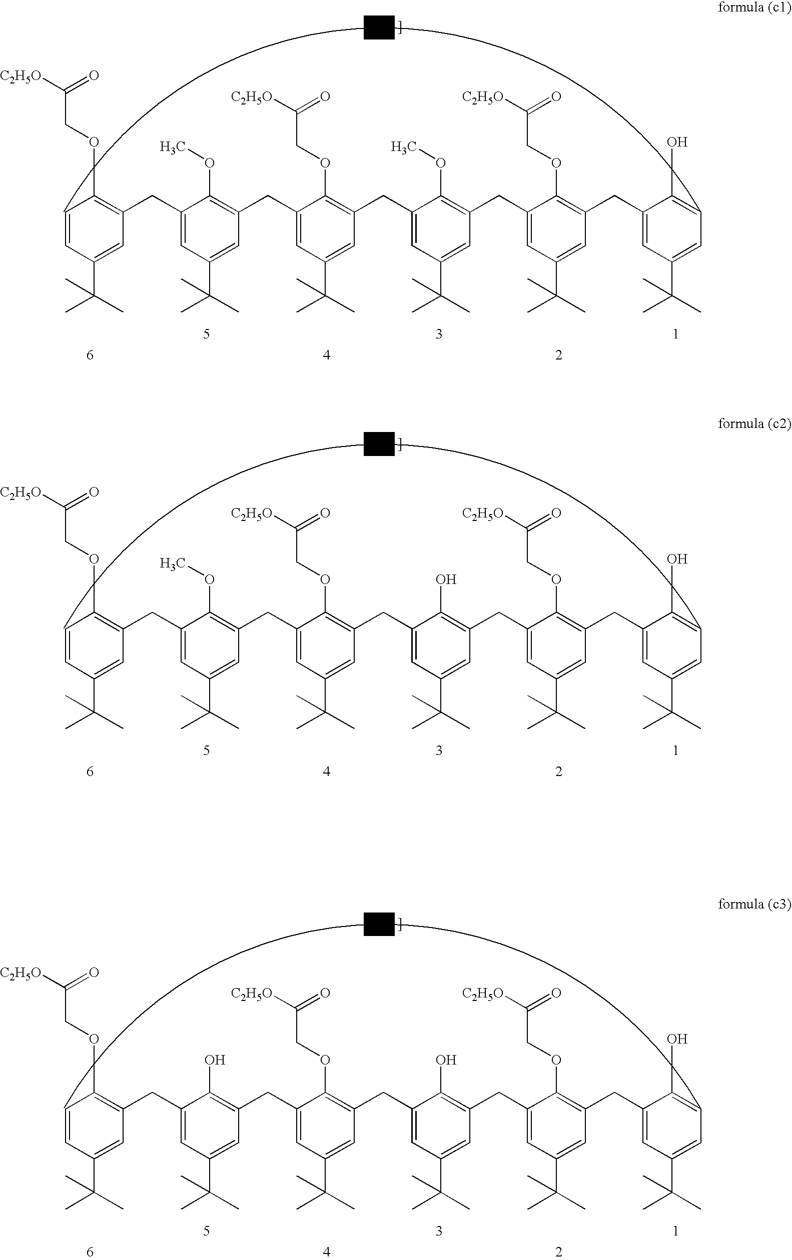

The compounds of formula (c1) or (c2) or (c3) are subsequently modified by chemical modification of the hydroxyls to result, on the one hand, in compounds of the general formula (IA), by saponification of the ethyl esters, or, on the other hand, in compounds of general formula (IB), by producing hydroxamic acid functional groups from the ethyl esters.

According to an alternative form of the process, the compounds of formula (c1) or (c2) or (c3) can subsequently be directly reacted with suitably functionalized supports to result in covalent grafting reactions via the activation of their free phenolic hydroxyls, in order to result, on the one hand, in support materials of general formula (IIA), by saponification of the ethyl esters, or, on the other hand, in support materials of general formula (IIB), by producing hydroxamic acid functional groups from the ethyl esters.

Thus, a support material in accordance with the invention can also be represented in the following way:

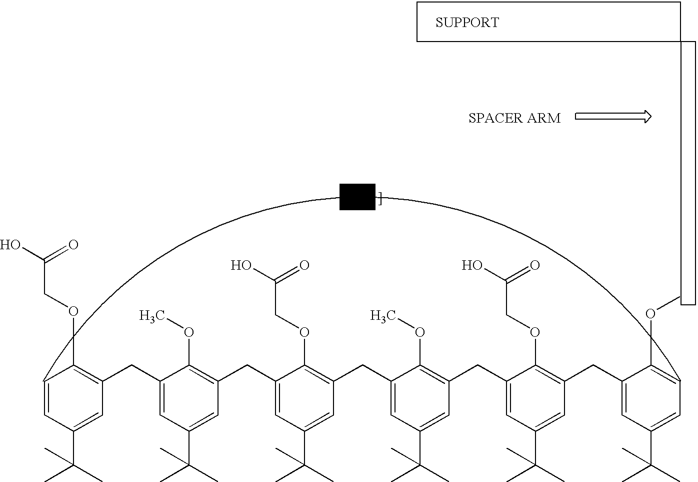

The details of the stages of this synthesis are given below:

### A) Synthesis of 1,3,5-trimethoxy-para-tert-butylcalix-[6]arene (RM=1014)

The process consists in synthesizing, in a first step, a common precursor comprising carboxylic acid ester functional groups in the 2, 4 and 6 positions and trimethoxy functional groups in the 1, 3 and 5 positions.

During this objective, it is first necessary to synthesize a symmetrical compound 1,3,5-trimethoxy-p-tert-butylcalix[6]arene (RM=1014) of formula (a) above.

Commercial para-tert-butylcalix[6]arene is dissolved in anhydrous acetone. Potassium carbonate is added and the suspension is stirred under nitrogen for 3 hours. Excess methyl iodide is then added and the reaction suspension is gradually brought to reflux for 24 hours with stirring.

The acetone is subsequently evaporated to dryness under the vacuum of a laminar flow pump on a water bath adjusted to 60° C. The solid residue obtained from the drying operation is dissolved in chloroform. After dissolution, water is added and the two-phase medium is placed under vigorous stirring and then acidified using 12M concentrated hydrochloric acid. The lower organic phase is subsequently recovered by separation by settling and then washed with water until the aqueous washing liquor present in the upper phase is neutral. The lower organic phase is subsequently dried over anhydrous sodium sulfate and then filtered. The clear organic phase is concentrated to dryness on a water bath at 60° C. under laminar flow vacuum. The drying residue is subsequently purified by low-pressure chromatography on silica gel of chromatographic grade. The eluent used is pure synthetic methylene chloride stabilized with amylene. The purity of the various fractions is monitored in methylene chloride/ethanol 95/5 by TLC on virgin silica plates.

The fractions comprising the symmetrical compound 1,3,5-trimethoxy-p-tert-butylcalix[6]arene, single spot by TLC (visualization by iodine vapor), are combined and then concentrated to dryness.

The residue is used as is in the following stage.

### B) Synthesis of 2,4,6-tri(ethyl ester)-1,3,5-trimethoxy-para-tert-butylcalix[6]arene (RM=1272.5) (Formula (b))

In a second step, the process consists in synthesizing the common precursor, comprising carboxylic acid ester functional groups in the 2, 4 and 6 positions and trimethoxy functional groups in the 1, 3 and 5 positions, by modifying all the phenolic hydroxyls of the compound obtained above which are located in the 2, 4 and 6 positions, the 1, 3 and 5 positions being protected by the methoxy groups.

The product obtained in the preceding example (formula (a)) is dissolved in anhydrous DMF (dimethylformamide) (dried over sodium hydride) under a nitrogen atmosphere. A very large excess of cesium carbonate is added and the suspension obtained is stirred under nitrogen for 4 hours. A very large excess of ethyl bromoacetate is subsequently run in over 5 minutes onto the reaction suspension and the vigorously stirred medium is gradually brought to reflux for 24 hours while bubbling nitrogen through.

The DMF is evaporated to dryness under the vacuum of a laminar flow pump on a water bath adjusted to 80° C. The solid residue obtained from the drying is dissolved in chloroform. After dissolving, water is added and the two-phase medium is placed under vigorous stirring and then acidified with 12M concentrated hydrochloric acid. The lower organic phase is subsequently washed several times with water and then it is dried over anhydrous sodium sulfate. After filtering, the organic phase is concentrated to dryness on a water bath at 60° C. under laminar flow vacuum. The drying residue is taken up in ethanol. A white suspension is obtained. The white solid is recovered by filtration. It is washed several times on the filter with ethanol and then dried in an oven at 40° C. under vacuum.

### C) Synthesis of 2,4,6-tri(ethyl ester)-1-hydroxy-3,5-dimethoxy-para-tert-butylcalix[6]arene (Compound (c1)) 2,4,6-tri(ethyl ester)-1,3-dihydroxy-5-monomethoxy-para-tert-butylcalix[6]arene (Compound (c2)) 2,4,6-tri(ethyl ester)-1,3,5-trihydroxy-para-tert-butylcalix[6]arene (Compound (c3))

In an optional third stage, the process consists in partially or completely demethylating the common precursor of formula (b), comprising carboxylic acid ester functional groups in the 2, 4 and 6 positions and both phenolic hydroxyl and methoxy functional groups in the 1, 3 and 5 positions (compounds (c1) or (c2)) or solely phenolic hydroxyl groups in the 1, 3 and 5 positions (compound (c3)).

To do this, the compound of formula (b) obtained above is dissolved in anhydrous chloroform dried beforehand over sodium hydride. The medium is stirred under a nitrogen atmosphere and then trimethylsilyl iodide (demethoxylating agent for calixarenes) is added, in stoichiometric amount or in deficit depending on whether it is desired to favor mono-, di- or tridemethylation, and the reaction medium is brought to reflux for 2 hours while bubbling nitrogen through. The reaction kinetics are determined by TLC monitoring in toluene/ethyl acetate 90/10 on a silica/polyester plate. Trimethylsilyl iodide is optionally again added all at once after cooling the reaction medium, depending on the kinetics of formation of the desired entities (molecules of formula (c1) or (c2) or (c3)). The reaction medium is again gradually brought to reflux for 2 hours or more.

The reaction is halted by the addition of water. The reaction medium is acidified with 1M HCl and the lower organic phase, which is brick red, is recovered by separation by settling. It is washed with water until the aqueous washing liquor (upper phase) is of neutral pH.

After drying over anhydrous sodium sulfate, the lower chloroform phase is evaporated to dryness under the vacuum of a laminar flow pump on a water bath adjusted to 60° C. Residue obtained from the drying is subsequently purified by low-pressure chromatography on silica gel of chromatographic grade. The eluent used is the toluene/ethyl acetate 90/10 mixture. The purity of the various fractions is monitored in the toluene/ethyl acetate 90/10 mixture by TLC on plates of virgin silica deposited on a polyester support.

The fractions comprising the compounds (c1) or (c2) or (c3) are combined and brought to dryness separately under the evaporation conditions described above.

Each residue from the drying operation is used as is for possible subsequent chemical modifications.

### D) Synthesis of 2,4,6-tri(ethyl ester)-1-R1-3,5-dimethoxy-para-tert-butylcalix[6]arene (Compound (d1)) 2,4,6-tri(ethyl ester)-1,3-R1,R3-5-monomethoxy-para-tert-butylcalix[6]arene (Compound (d2)) 2,4,6-tri(ethyl ester)-1,3,5-R1,R3,R5-para-tert-butylcalix[6]arene (Compound (d3))

In another optional stage, the process consists in synthesizing a compound of general formula (d1) or (d2) or (d3) from respectively the compounds (c1) or (c2) or (c3) obtained above. The phenolic hydroxyl functional groups in the 1 and/or 3 and/or 5 positions are modified and R1 and/or R3 and/or R5 groups are introduced, it being known that they cannot represent hydroxyls or methyls.

To do this, a compound of formula (c1) or (c2) or (c3) obtained above is dissolved in anhydrous DMF (dimethyl-formamide) (dried over sodium hydride) under a nitrogen atmosphere. A very large excess of cesium carbonate is added and the suspension obtained is stirred under nitrogen for 4 hours. A very large excess of halide, the organic residue of which represents the R1 or R3 or R5 group, is subsequently run in in 5 minutes onto the reaction suspension and the vigorously stirred medium is gradually brought to reflux for 24 hours while bubbling nitrogen through.

The DMF is evaporated to dryness under the vacuum of a laminar flow pump on a water bath adjusted to 80° C. The solid residue obtained from the drying is dissolved in chloroform. After dissolving, water is added and the two-phase medium is placed under vigorous stirring and then acidified with 12M concentrated hydrochloric acid. The low organic phase is subsequently washed several times with water and then it is dried over anhydrous sodium sulfate. After filtering, the organic phase is concentrated to dryness on a water bath at 60° C. under laminar flow vacuum. The drying residue is taken up in chloroform.

After drying over anhydrous sodium sulfate, the lower chloroform phase is evaporated to dryness under the vacuum of a laminar flow pump on a water bath adjusted to 60° C. The drying residue obtained is subsequently purified by low-pressure chromatography on silica gel of chromatographic grade. The eluent used is the toluene/ethyl acetate 90/10 mixture. The purity of the various fractions is monitored in the toluene/ethyl acetate 90/10 mixture by TLC on plates of virgin silica deposited on a polyester support.

The fractions comprising the compound (d1) or (d2) or (d3) are combined and brought to dryness separately under the evaporation conditions described above.

The drying residue is used as is for possible subsequent chemical modifications.

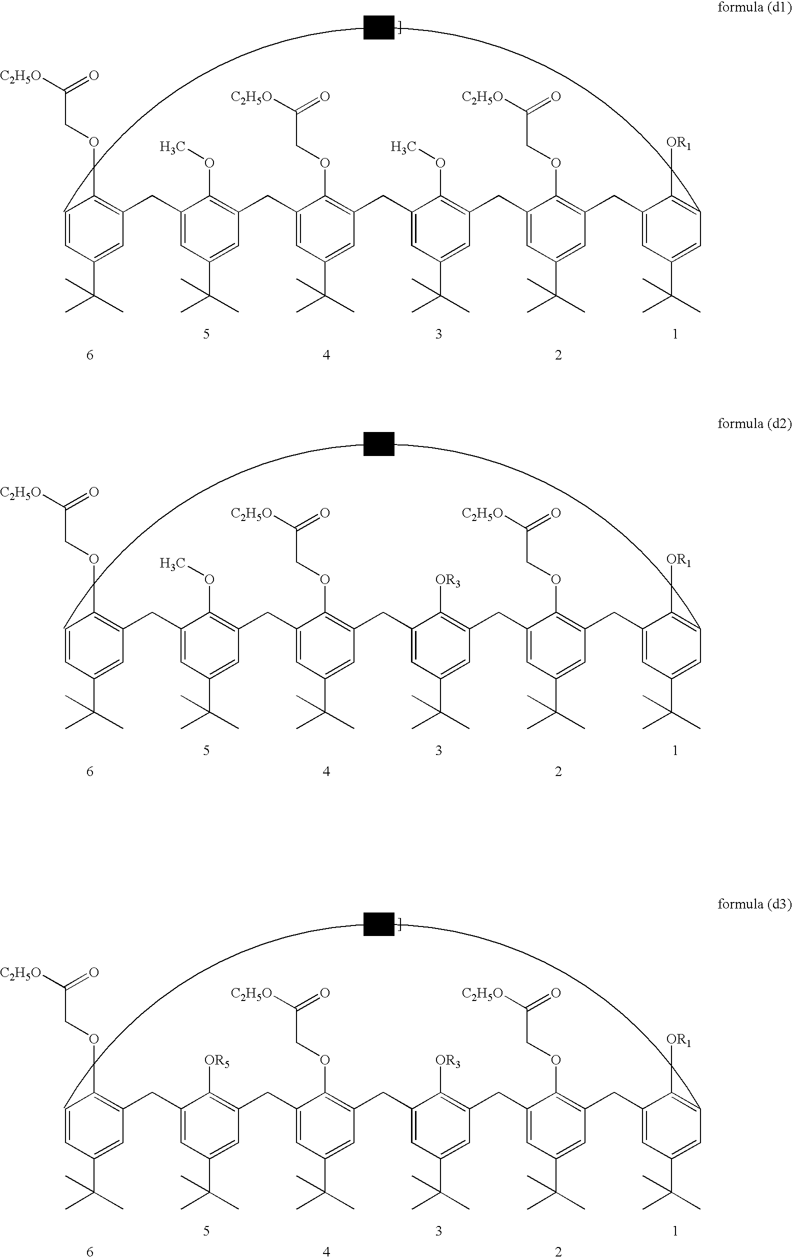

### E) Grafting to a Support of One of the Following Compounds: (c1), (c2), (c3), (d1), (d2), (d3)

In another optional stage, the process consists in covalently grafting, to a support, a compound of formula (c1) or (c2) or (c3) or (d1) or (d2) or (d3) comprising carboxylic acid ester functional groups in the 2, 4 and 6 positions and both phenolic hydroxyl and methoxy functional groups in the 1 and/or 3 positions (compounds (c1) or (c3)) or solely phenolic hydroxyl groups in the 1, 3 and 5 positions (compound (c3)), or R1 and/or R3 and/or R5 functional groups (where R1, R3 and R5 are other than hydrogen and methyl) in the 1, 3 or 5 positions (compounds (d1), (d2) and (d3)).

A compound of formula (c1) or (c2) or (c3) or (d1) or (d2) or (d3) is dissolved in anhydrous dimethylformamide dried beforehand over sodium hydride. The medium is stirred under nitrogen until dissolution is complete. A very large excess of cesium carbonate is added, followed by a commercial resin of known and quantified functionality, said functionality being chosen in order to be able to react with a phenolic hydroxyl or with an R1 and/or R3 and/or R5 group, and the reaction medium is brought to 60° C. for 72 hours while bubbling nitrogen through.

The reaction suspension is filtered and the solid is washed with DMF, then with acetone and then with 1M HCl until the aqueous washing liquor is of acidic pH. The resin is subsequently washed with water to neutrality and then with ethanol.

### F) Synthesis of a Compound of General Formula IIA

In one option of the process, use is made of a compound of general formula (a) or (c1) or (c2) or (c3) or (d1) or (d2) or (d3) or a compound obtained by using the process described in part E.

Said compound is dissolved or suspended in ethanol. An aqueous potassium hydroxide solution, in very large excess with respect to the stoichiometry calculated with regard to the number of ethyl ester groups to be saponified, is added and the medium (solution or suspension) is brought to reflux for 4 hours.

The reaction mass is cooled and the medium is acidified with 12M HCl.

The suspension is filtered and the precipitate (or the support) is washed with water until the aqueous filtration liquors are of neutral pH and then with ethanol.

The solid is dried at 40° C. under vacuum to constant weight.

### G) Synthesis of a Compound of Formula IIB

In one option of the process, use is made of a compound of general formula (a) or (c1) or (c2) or (c3) or (d1) or (d2) or (d3) or a compound obtained by using the process described in part E.

Said compound is dissolved or suspended in THF. A methanolic hydroxylamine hydrochloride solution, in excess with respect to the stoichiometry calculated with regard to the number of the ethyl ester groups, is added. A second solution, comprising potassium hydroxide flakes dissolved beforehand in a methanol/THF mixture and maintained at ±5° C., is then added. The medium (solution or suspension) is stirred at ambient temperature for 7 days under a nitrogen atmosphere.

The reaction mass is evaporated to dryness on a water bath at 60° C. under vacuum.

The residue is taken up in methylene chloride and then acetic acid is added. The medium is stirred at 20-25° C. for 4 hours and then it is again dried.

The reaction mass is evaporated to dryness on a water bath at 60° C. under vacuum.

The invention also relates to the use of a supported liquid membrane as described above and/or of a support material according to the invention for the selective complexing and the analysis of the elements uranium, americium and plutonium or other radioelements in their cationic form.

The invention also relates to the use of a supported liquid membrane and/or of a support material according to the invention for removing, from a mixture of at least two constituents chosen from the group comprising of organic, inorganic and organo-inorganic molecules, at least a portion of one of these constituents or for separating said constituents by a chromatographic method.

The supported liquid membranes and the support materials of the invention are particularly suitable for exclusion chromatography. They make possible the detection and separation of minute amounts of product, in particular the separation of uranium and/or plutonium and/or americium at contents of the order of 1 mBq/l.

The invention will be described in more detail below with the help of the following examples, which are given by way of illustration and are not limiting.

### EXAMPLES

### Example 1

### Support Material Comprising 3,5-dimethoxy-2,4,6-tri(carboxylic acid)-p-tert-butylcalix[6]arene

### 1-1: Synthesis of 1,3,5-trimethoxy-p-tert-butylcalix[6]arene (RM=1014)

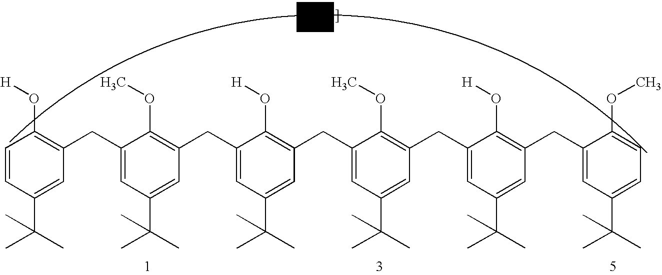

194.7 g of p-tert-butylcalix[6]arene (0.2 mol, RM=972) and 15 l of anhydrous acetone are introduced into a 20-liter glass reactor equipped with a condenser. The medium is stirred under nitrogen until dissolution is complete. 82.9 g of potassium carbonate (0.6 mol) are added and the suspension is stirred for 3 hours under nitrogen. 113.6 g of methyl iodide (0.8 mol) are run in in 5 minutes and the stirred reaction medium is gradually brought to reflux for 24 hours.

The acetone is evaporated to dryness under the vacuum of a laminar flow pump on a water bath adjusted to 60° C. The solid residue obtained from the drying is dissolved in 5.0 l of chloroform. After dissolving, 1.0 l of water is added and the two-phase medium is placed under vigorous stirring. 0.1 l of 12M concentrated hydrochloric acid is gently added according to the release of carbon dioxide gas. The organic phase is subsequently washed 5 times with 1.0 l of water and then dried over 200 g of anhydrous sodium sulfate. After filtering, the organic phase is concentrated to dryness on a water bath at 60° C. under laminar flow vacuum. 250 g of drying residue are obtained and are subsequently purified by low-pressure chromatography on 15 kg of 40-200 μm silica gel (60 Å pore). The eluent used is methylene chloride (40 l). The purity of the various fractions is monitored in methylene chloride/ethanol 95/5 by TLC on virgin silica plates.

40 g of residue, resulting from the fractions comprising pure 1,3,5-trimethoxycalixarene, are obtained (39.45 mmol: yield=19.7%).

The proton NMR spectrum in CDCl₃ gives the following chemical shifts:

7.00 ppm (s, 6H, ArH meta OH), 6.90 ppm (s, 6H, ArH meta OCH₃), 6.75 ppm (s, 3H, OH), 3.89 ppm (s, 9H, OCH₃), 3.47 ppm (s, 12H, ArCH₂Ar), 1.20 ppm (s, 27H, tert-butyl para OH), 1.00 ppm (s, 27H, tert-butyl para OCH₃).

### 1-2: Synthesis of 2,4,6-tri(ethyl ester)-1,3,5-trimethoxy-p-tert-butylcalix[6]arene (RM=1272.5)

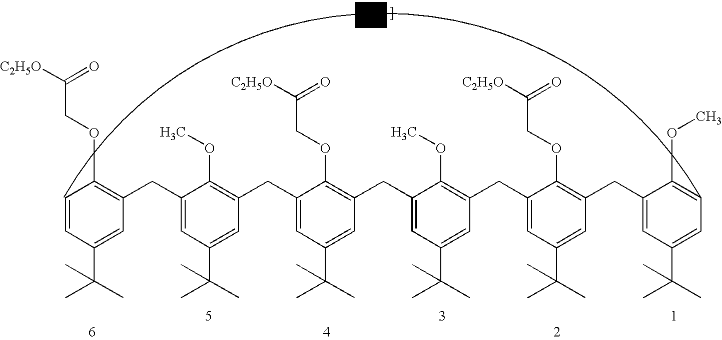

40.0 g of product obtained in 1-1 (39.45 mmol) and 4 l of anhydrous dimethylformamide (DMF) (dried over sodium hydride) are introduced into a 10-liter glass reactor equipped with a condenser. The medium is stirred under nitrogen until dissolution is complete. 81.6 g of cesium carbonate (0.25 mol) are added and the suspension is stirred for 4 hours under nitrogen. 52.7 g of ethyl bromoacetate (0.31 mol) are run in in 5 minutes and the stirred reaction medium is gradually brought to reflux for 24 hours while bubbling nitrogen through.

The DMF is evaporated to dryness under the vacuum of a laminar flow pump on a water bath adjusted to 80° C. The solid residue obtained from the drying is dissolved in 2.0 l of chloroform. After dissolving, 0.5 l of water are added and the two-phase medium is placed under vigorous stirring. 20 ml of 12M concentrated hydrochloric acid are gently added as a function of the release of carbon dioxide gas. The organic phase is subsequently washed 5 times with 0.5 l of water and then dried over 20 g of anhydrous sodium sulfate. After filtering, the organic phase is concentrated to dryness on a water bath at 60° C. under laminar flow vacuum. The residue is taken up in 300 ml of ethanol. A white suspension is obtained. The solid is recovered by filtration, washed with 3 times 40 ml of ethanol and then dried in an oven at 40° C. under vacuum. 43.2 g of white powder are obtained after drying to constant weight (33.95 mmol: yield=86%).

The proton NMR spectrum in CDCl₃ gives the following chemical shifts:

6.71 ppm (broad s, 12H, ArH meta), 4.55 ppm (s, 6H, ArCH₂CO₂R), 4.29 ppm (qt, 6H, O—CH₂-methyl J=7 Hz), 3.47 ppm (s, 21H, ArCH₂Ar+methyl OCH₃), 1.38 ppm (s, 54H, tert-butyl), 1.33 ppm (t, 9H, methyl OCH₂CH₃ J 7 Hz).

### 1-3: Synthesis of 1-hydroxy-3,5-dimethoxy-2,4,6-tri(ethyl ester)-p-tert-butylcalix[6]arene (RM=1258.5)

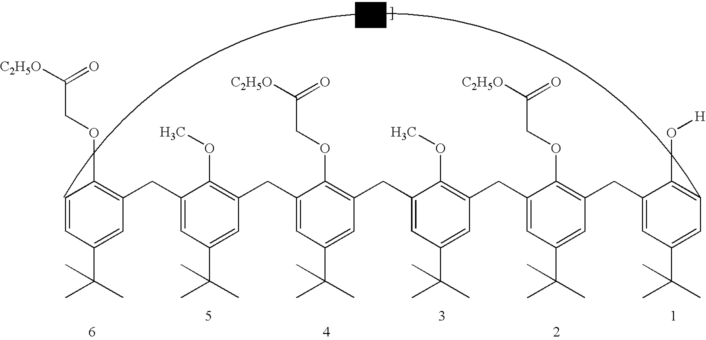

27.8 g of product (white powder) obtained in 1-2 above (21.85 mmol, RM=1272.5) and 1.5 l of anhydrous chloroform dried beforehand over sodium hydride are introduced into a 5-liter glass reactor equipped with a condenser. The medium is stirred under nitrogen until dissolution is complete. 3.1 ml (4.37 g) of trimethylsilyl iodide (21.85 mmol, RM=200.1) are added and the reaction medium is brought to reflux for 2 hours while bubbling nitrogen through. The reaction kinetics are determined by TLC monitoring in toluene/ethyl acetate 90/10 on a silica/polyester plate. 3.1 ml (4.37 g) of trimethylsilyl iodide are again added all at once after cooling the reaction medium. The latter is again gradually brought to reflux for 2 hours.

The reaction is halted by addition of 2 l of water. 100 ml of 1M HCl are added and the brick red organic phase is recovered. It is washed with 2 times 1 l of water.

After drying with 200 g of anhydrous sodium sulfate, the chloroform phase is evaporated to dryness under the vacuum of a laminar flow pump on a water bath adjusted to 60° C. 27.2 g of drying residue are obtained and are subsequently purified by low-pressure chromatography on 3 kg of 40-200 μm silica gel (60 Å pore). The eluent used is a toluene/ethyl acetate 90/10 mixture (20 l). The purity of the various fractions is monitored in the toluene/ethyl acetate 90/10 mixture by TLC on plates of virgin silica deposited on a polyester support.

8.5 g of residue are obtained (6.75 mmol: yield=30.7%) after drying, on a water bath at 60° C. under vacuum, the fractions comprising the pure 1-hydroxy-3,5-dimethoxy-2,4,6-tri(ethyl ester)-p-tert-butylcalix[6]arene.

The mass spectrum, recorded in FAB with positive chemical ionization, confirms the presence of the expected product (MH+ at 1259 daltons).

The proton NMR spectrum in CDCl₃ gives the following chemical shifts:

6.75 ppm (s, 13H, ArH meta+OH phenol), 4.55 ppm (s, 6H, ArCH₂CO₂R), 4.29 ppm (qt, 6H, O—CH₂-methyl J=7 Hz), 3.47 ppm (s, 20H, ArCH₂Ar+methyl OCH₃), 1.38 ppm (s, 54H, tert-butyl), 1.33 ppm (t, 9H, methyl OCH₂CH₃).

### 1-4: Support Material Comprising 3,5-dimethoxy-2,4,6-tri(ethyl ester)-p-tert-butylcalix[6]arene

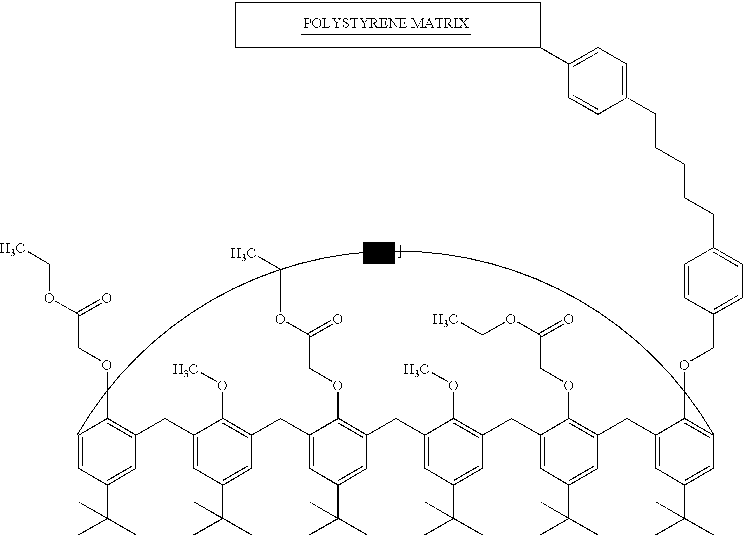

8.5 g of product obtained in 1-3 above (6.75 mmol, RM=1258.5) and 150 ml of anhydrous DMF dried beforehand over sodium hydride are introduced into a 250 ml glass reactor equipped with a condenser. The medium is stirred under nitrogen until dissolution is complete. 20 g of cesium carbonate (61 mmol) are added, followed by 10 g of commercial polystyrene resin modified with chloromethylphenylpentyl (purchased from Aldrich CMPP resin: [5-[4-(chloromethyl)phenyl]pentyl]styrene, polymer bound reference 513776), and the reaction medium is brought to 60° C. for 72 hours while bubbling nitrogen through.

The reaction suspension is filtered and the solid is washed with DMF (2 times 50 ml), then with acetone (3 times 50 ml), then with 1M HCl (4 times 100 ml), then with water (5 times 100 ml) and then with ethanol (3 times 50 ml).

16.15 g of dry resin are obtained after drying at 60° C. under vacuum to constant weight.

Its degree of grafting, calculated from the microanalysis, is 0.2 mmol of calix/g of resin.

The microanalysis is as follows:

C %: 80.80H %: 7.29 Cl %: 0.79

The microanalysis of the starting chloromethylphenyl-pentyl resin was as follows:

C %: 86.52H %: 7.87 Cl %: 3.91

### 1-5: Support Material Comprising 3,5-dimethoxy-2,4,6-tri(carboxylic acid)-p-tert-butylcalix[6]arene

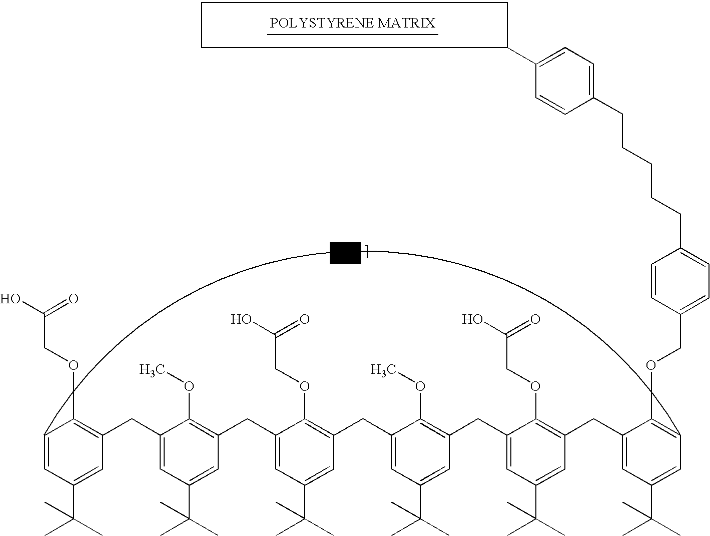

10 g of dry resin obtained in 1-4 above and 100 ml of ethanol are introduced into a 250 ml glass reactor equipped with a condenser. 6.1 g of 85% potassium hydroxide pellets are dissolved in 100 ml of water and the solution obtained is added all at once to the reactor. The reaction medium is brought to reflux for 4 hours while bubbling nitrogen through.

The reaction suspension is cooled and then 12 ml of 12M HCl are added. The pH of the suspension is 1. After stirring for 1 hour, the suspension is filtered and then the filter residue is washed with 8 times 100 ml of water and then with ethanol (3 times 50 ml).

9.7 g of resin are obtained after drying at 60° C. under vacuum to constant weight.

The resin is used as is without additional analytical characterization.

### Example 2

### Supported Liquid Membrane Comprising 1,3,5-trimethoxy-2,4,6-tri(hydroxamic acid)-p-tert-butylcalix[6]arene (RM=1234.6)

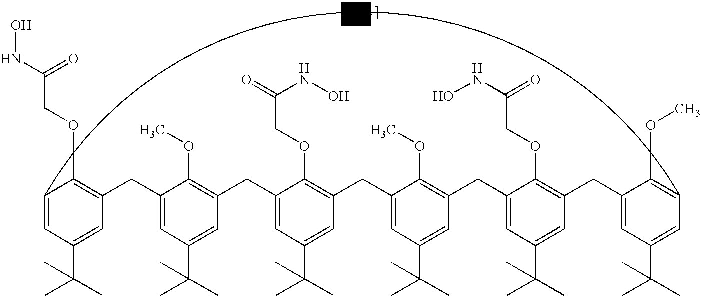

1.8 g of product obtained in example 1-2 (1.4 mmol) and 50 ml of tetrahydrofuran (THF) are introduced into a 250 ml glass reactor equipped with a condenser. 2.02 g of hydroxylamine hydrochloride (29 mmol) are dissolved in 80 ml of methanol and 40 ml of THF and then the solution obtained is added to that present in the reactor. Subsequently, another solution prepared beforehand (and maintained at +5° C.) of potassium hydroxide flakes (2.04 g at 100%, i.e. 36 mmol) in 24 ml of methanol and 12 ml of THF is added all at once to the reactor. The reaction medium is stirred at ambient temperature for 7 days while bubbling nitrogen through.

The reaction suspension is evaporated to dryness on a water bath at 60° C. under vacuum. The residue is taken up in the methylene chloride/acetic acid 50 ml/10 ml mixture and stirred for 4 hours. The reaction mass is brought to dryness on a water bath at 60° C. under vacuum.

The drying residue is taken up in 20 ml of dichloromethane. The expected product precipitates in the form of a white solid.

1.5 g of white solid are obtained after drying at 60° C. under vacuum to constant weight (yield=85.9%).

The mass spectrum, recorded in electrospray ESI, confirms the presence of the expected product (m/z at 1234 daltons).

The proton NMR spectrum at 300 MHz in DMSO gives the following chemical shifts:

10.8 ppm (s, 3H, —NH), 9.08 ppm (s, 3H, —OH of hydroxamic), 7.23 ppm (s, 6H, ArH meta OCH₂COOEt), 6.57 ppm (s, 6H, ArH meta OCH₃), 4.44 to 4.33 ppm (q, 18H, ArCH₂CO₂R+ArCH₂Ar), 2.50 ppm (broad s, 9H, methoxy), 1.36 ppm (s, 27H, tert-butyl para OCH₂CONHOH), 0.73 ppm (s, 27H, tert-butyl para OCH₃).

2 g of resin, Macroprep epoxy, from BioRad, particle size of 70 to 100 μm, batch 11/99, are added to the solution prepared beforehand of 15.6 mg of white solid obtained above, withdrawn from the 1.5 g of white solid obtained beforehand, dissolved in 20 ml of dichloromethane and 1.12 ml of 1,2,3,4-tetrahydro-naphthalene. The suspension is gently evaporated at ambient temperature to constant weight: a white solid is obtained.

Weight obtained: 2.2 g

### Example 3

### Supported Liquid Membrane Comprising 1,3,5-trimethoxy-2,4,6-tri(carboxylic acid)-p-tert-butylcalix[6]arene (RM=1174.4)

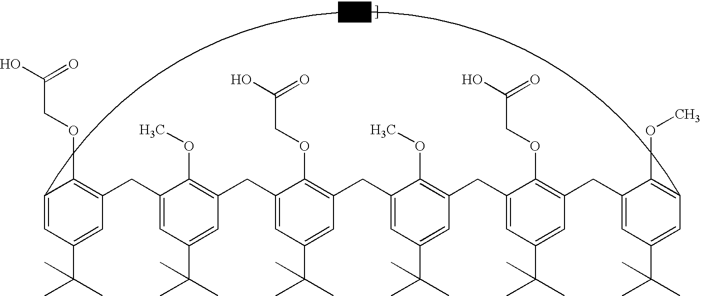

5.9 g of product (white powder) obtained in example 1-2 (4.00 mmol) and 150 ml of ethanol are introduced into a 250 ml glass reactor equipped with a condenser. A solution prepared beforehand (and maintained at +5° C.) of potassium hydroxide flakes (12 g at 100%, i.e. 214 mmol) in 150 ml of water is added all at once to the reactor. The reaction medium is brought to reflux for 4 hours while bubbling nitrogen through.

25 ml of 12M HCl are slowly added after cooling the reaction mass to 20° C. The expected product precipitates in the form of a white solid. A suspension is subsequently filtered and the solid is washed with 8 times 50 ml of water and then 2 times 50 ml of ethanol. The solid is subsequently dried at 40° C. under vacuum to constant weight.

4.2 g of white solid are obtained after drying at 40° C. under vacuum to constant weight (yield=99%).

The proton NMR spectrum at 300 MHz in CDCl₃ gives the following chemical shifts:

6.97 ppm (s, 6H, ArH meta OCH₂COOH), 6.94 ppm (s, 6H, ArH meta OCH₃), 3.84 ppm (s, 6H, ArCH₂COOH), 3.73 ppm (broad s, 9H, methoxy), 1.12 ppm (s, 27H, tert-butyl para OCH₂COOH), 1.09 ppm (s, 27H, tert-butyl para OCH₃).

2 g of resin, Macroprep epoxy, from BioRad, particle size of 70 to 100 μm, batch 11/99, are added to the solution prepared beforehand of 12.5 mg, withdrawn from 4.2 g of white solid obtained above, dissolved in 20 ml of dichloromethane and 1.12 ml of 1,2,3,4-tetrahydro-naphthalene. The suspension is gently evaporated at ambient temperature to constant weight. A white solid is obtained.

Weight obtained: 2.2 g

### Example 4

### Selective Complexing and Extracting of Americium

In this example, the americium present at a concentration of 10⁻¹¹ mol.l⁻¹ in a 0.04 mol.l⁻¹ aqueous NaNO₃ solution simulating the urinary medium and adjusted to pH=4 is fixed using the calixarenes of the invention.

For this experiment, use is made of 100 mg of support material from example 1-5 packaged in a column.

A 0.04 mol.l⁻¹ NaNO₃ solution at pH=4 is passed through the column in order for optimum extraction conditions to exist (conditioning stage). The aqueous solution comprising the americium is subsequently passed through the column (fixing stage). A 0.04 mol.l⁻¹ NaNO₃ solution at pH=4 is again passed in order to remove the americium not extracted by the calixarene (rinsing stage). The fixed americium is finally eluted with a 2M HNO₃ solution (elution stage). The solutions flow by natural gravitation. The americium is measured by alpha spectrometry in each solution at the column bottom. The results of the measurements make it possible to calculate the fixing yield and the elution yield for the americium.

The results of this experiment are given in table I below.

### Example 5

### Selective Complexing and Extracting of Uranium

In this example, the uranium present at a concentration of 10⁻⁸ mol.l⁻¹ in a 0.04 mol.l⁻¹ aqueous NaNO₃ solution simulating the urinary medium and adjusted to pH=4 is fixed by means of the calixarenes of the invention.

For this experiment, use is made of 1 g of supported liquid membrane from example 2 packaged in a column.

The conditioning, fixing and rinsing stages are identical to those described in example 4. The fixed uranium is eluted with a 1M HNO₃ solution. The uranium is measured by alpha spectrometry or by mass spectrometry (ICP-MS) in each solution at the column bottom. The results of the measurements make it possible to calculate the fixing yield and the elution yield for the uranium.

The results obtained are given in table I below.

### Example 6

### Selective Complexing and Extracting of Uranium in Urine

In this example, the uranium present at a concentration of 5.10⁻⁶ g/l in urine is fixed by means of the calixarenes of the invention.

For this experiment, use is made of 1 g of supported liquid membrane from example 3 packaged in a column.

A preliminary stage of mineralization of the urine is carried out by heating using microwave radiation. The mineralization residue is taken up in a 2M HNO₃ solution and then the pH of the solution is adjusted to 4 before passing through the column.

The conditioning, fixing, rinsing and elution stages are identical to those described in example 4. The uranium is measured and the results are expressed as above.

The results obtained are given in table I below.

### Example 7

### Selective Complexing and Extracting of Plutonium

In this example, the plutonium present at a concentration of 10⁻¹⁰ mol.l⁻¹ in a 0.04 mol.l⁻¹ aqueous NaNO₃ solution simulating the urinary medium and adjusted to pH=4 is fixed by means of the calixarenes of the invention.

For this experiment, use is made of 100 mg of support material from example 1-5 packaged in a column.

The conditioning, fixing, rinsing and elution stages are identical to those described in example 4. The plutonium is measured by alpha spectrometry or by mass spectrometry (ICP-MS) in each solution at the column bottom. The results of the measurements make it possible to calculate the fixing yield and the elution yield for plutonium.

The results obtained are given in table I below.

TABLE I
Fixing and elution yields (%)

| | Fixing yield (%) | Elution yield (%) |
|---|---|---|
| Example 4 | 97 ± 1 | 94 ± 9 |
| Example 5 | 99 ± 1 | 93 ± 1 |
| Example 6 | 98 ± 1 | 102 ± 3 |
| Example 7 | 83 ± 1 | 87 ± 6 |

### Example 8

### Supported Liquid Membrane Comprising 1-hydroxy-3,5-dimethoxy-2,4,6-tri(carboxylic acid)-p-tert-butyl-calix[6]arene (RM=1174.4)

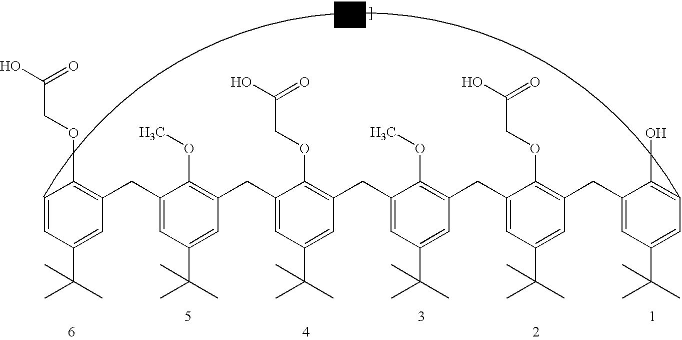

5.03 g of product (white powder) obtained in example 1-3, during the implementation of a second test identical to the preceding one, (4.00 mmol) and 150 ml of ethanol are introduced into a 250 ml glass reactor equipped with a condenser. A solution prepared beforehand (and maintained at +5° C.) of potassium hydroxide flakes (12 g at 100%, i.e. 214 mmol) in 150 ml of water is added all at once to the reactor. The reaction medium is brought to reflux for 4 hours while bubbling nitrogen through.

25 ml of 12M HCl are slowly added after cooling the reaction mass to 20° C. The expected product precipitates in the form of a white solid. The suspension is subsequently filtered and the solid is washed with 8 times 50 ml of water and then 2 times 50 ml of ethanol. The solid is subsequently dried at 40° C. under vacuum to constant weight.

4.65 g of white solid are obtained after drying at 40° C. under vacuum to constant weight (yield=99%).

The proton NMR spectrum at 300 MHz in CDCl₃ gives the following chemical shifts:

6.86 ppm (s, 13H, ArH meta+OH phenol), 3.95 ppm (s, 6H, ArCH₂COOH), 3.73 ppm (broad s, 6H, methoxy), 1.12 ppm (s, 27H, tert-butyl para OCH₂COOH), 1.09 ppm (s, 27H, tert-butyl para OCH₃).

2 g of resin, Macroprep epoxy, from BioRad, particle size of 70 to 100 μm, batch 11/99, are added to the solution prepared beforehand of 12.5 mg, withdrawn from the 4.65 g of white solid obtained above, dissolved in 20 ml of dichloromethane and 1.12 ml of 1-heptanol. The suspension is gently evaporated at ambient temperature to constant weight: a white solid is obtained.

Weight obtained: 2.2 g

### Selective Complexing and Extracting of Uranium

In this example, the uranium present at a concentration of 10⁻⁸ mol.l⁻¹ in a 0.04 mol.l⁻¹ aqueous NaNO₃ solution simulating the urinary medium and adjusted to pH=4 is fixed by means of the calixarenes of the invention.

For this experiment, use is made of 1 g of supported liquid membrane obtained above packaged in a column.

The conditioning, fixing, rinsing and elution stages are identical to those described in example 4. The uranium is measured and the results are expressed as in the preceding examples.

The results obtained are given in table II below.

### Selective Complexing and Extracting of Thorium

In this example, the thorium present at a concentration of 10⁻⁸ mol.l⁻¹ in a 0.04 mol.l⁻¹ aqueous NaNO₃ solution simulating the urinary medium and adjusted to pH=3 is fixed by means of the calixarenes of the invention.

For this experiment, use is made of 1 g of the supported liquid membrane obtained above packaged in a column.

The conditioning, fixing, rinsing and elution stages are identical to those described in example 4, the conditioning and rinsing solutions being adjusted to pH 3. The thorium is measured by alpha spectrometry or by mass spectrometry (ICP-MS) in each solution at the column bottom and the results are expressed as in the preceding examples.

The results obtained are given in table II below.

TABLE II
Fixing and elution yields (%)

| Example 8 | Fixing yield (%) | Elution yield (%) |
|---|---|---|
| Uranium | 99 ± 1 | 73 ± 8 |
| Thorium | 60 ± 1 | 70 ± 1 |

1-13. (canceled)

14. A para-tert-butylcalix[6]arene of formula (IA) or (IB) 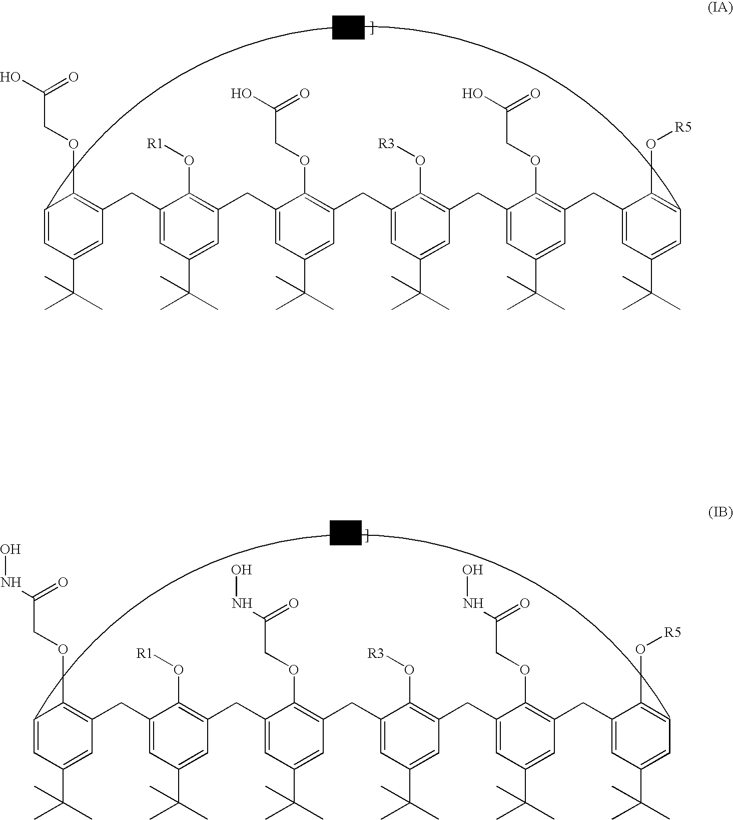
where R1, R3 and R5, which are identical or different, represent, each independently:
(i) a hydrogen or halogen atom,
(ii) an acetyl, amino, phosphate, nitro, sulfate, carboxyl, carboxylic, thiocarboxyl, carbamate or thiocarbamate radical,
(iii) an optionally substituted linear or branched alkyl having 1 to 60, preferably 1 to 30, carbon atoms which optionally exhibits at least one ethylenic or acetylenic unsaturation,
(iv) an optionally substituted cycloalkyl having from 3 to 12 carbon atoms which optionally exhibits at least one ethylenic or acetylenic unsaturation,
(v) an optionally substituted aryl, an optionally substituted naphthyl, an optionally substituted aryl(C₁-C₃₀ alkyl) or an optionally substituted (C₁-C₃₀ alkyl)aryl;
it being possible for the radicals (ii) to (v) to be substituted by halogen atoms, organometallic compounds, alcohol, amine, carboxylic, sulfonic, sulfuric, phosphoric, phosphonic or hydroxaniic acid or ester, carbamate, thiocarbamate, ether, thiol, epoxide, thioepoxide, isocyanate or isothiocyanate functional groups or it being possible for a carbon of these radicals to be replaced by a nitrogen, sulfur, phosphorus, oxygen, boron or arsenic heteroatom;
(vi) a polymer chosen from the group comprising of polystyrenes, copolymers of chloro- and/or bromomethylstyrene and of divinylbenzene, polyethers, polyacrylamides, poly(glycidyl methacrylate)s, dextraris and agaroses;
with the following conditions:
R1, R3 and R5 not simultaneously representing CH₃ in (IA) and (IB),
R1, R3 and R5 not simultaneously representing CH₂COOH in (IA), and R1, R3 and R5 not simultaneously representing CH₂CONHOH in (IB).

15. The para-tert-butylcalix[6]arene as claimed in claim 14, wherein two from R1, R3 and R5 represent hydrogen or methyl, the third being chosen from (vi) a polymer chosen from the group comprising of polystyrenes, copolymers of chloro- and/or bromomethylstyrene and of divinylbenzene, polyethers, polyacrylamides, poly(glycidyl methacrylate)s, dextrans and agaroses.

16. The para-tert-butylcalix[6]arene as claimed in 14, wherein R1, R3 and R5 are identical.

17. The para-tert-butylcalix[6]arene as claimed in 14, wherein R1, R3 and R5 represent a hydrogen.

18. A supported liquid membrane comprising a paratert-butylcalix[6]arene of formula (IA) or (IB) 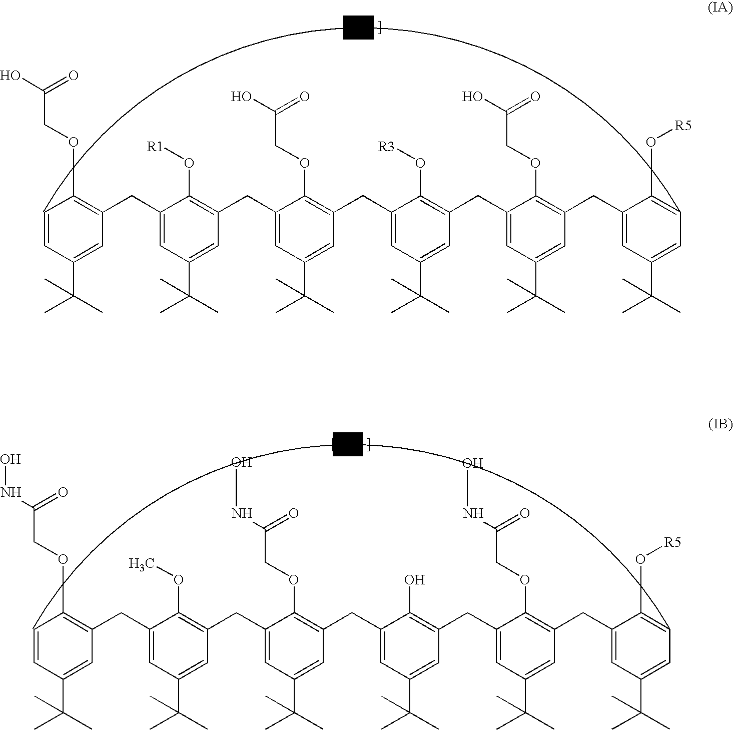
where R1, R3 and R5, which are identical or different, represent, each independently:
(i) a hydrogen or halogen atom,
(ii) an acetyl, amino, phosphate, nitro, sulfate, carboxyl, carboxylic, thiocarboxyl, carbainate or thiocarbamate radical,
(iii) an optionally substituted linear or branched alkyl having 1 to 60, preferably 1 to 30, carbon atoms which optionally exhibits at least one ethylenic or acetylenic unsaturation,
(iv) an optionally substituted cycloalkyl having from 3 to 12 carbon atoms which optionally exhibits at least one ethylenic or acetylenic unsaturation,
(v) an optionally substituted aryl, an optionally substituted naphthyl, an optionally substituted aryl(C₁-C₃₀ alkyl) or an optionally substituted (C₁-C₃₀ alkyl)aryl;
it being possible for the radicals (ii) to (v) to be substituted by halogen atoms, organometallic compounds, alcohol, amine, carboxylic, sulfonic, sulfuric, phosphoric, phosphonic or hydroxamic acid or ester, carbamate, thiocarbamate, ether, thiol, epoxide, thioepoxide, isocyanate or isothiocyanate functional groups or it being possible for a carbon of these radicals to be replaced by a nitrogen, sulfur, phosphorus, oxygen, boron or arsenic heteroatom;
(vi) a polymer chosen from the group comprising of polystyrenes, copolymers of chloro- and/or bromomethylstyrene and of divinylbenzene, polyethers, polyacrylamides, poly(glycidyl methacrylate)s, dextrans and agaroses;
said product of formula (IA) or (IB) being dissolved in an organic solvent and absorbed on a support.

19. A supported liquid membrane comprising a paratert-butylcalix{6]arene as claimed in claim 15.

20. The supported liquid membrane as claimed in claim 18, wherein the organic solvent exhibits a boiling point of greater than 60° C. and is chosen from the group comprising in particular toluene, xylene, chlorobenzene, ortho-dichlorobenzene, nitrobenzene, 1,4-diisopropylbenzene, hexylbenzene, kerosene, tetrahydropyran, 1,2,3,4-tetrahydronaphthalene, pentanol and higher homologous alcohols, glycols and their ethers, such as, for example, diethylene glycol dibutyl ether, esters, such as methyl benzoate, ethers, such as ortho-nitrophenyl pentyl ether or nitrophenyl octyl ether, and their mixtures.

21. The supported liquid membrane as claimed in claim 19, wherein the organic solvent exhibits a boiling point of greater than 60° C. and is chosen from the group comprising in particular toluene, xylene, chlorobenzene, ortho-dichlorobenzene, nitrobenzene, 1,4-diisopropylbenzene, hexylbenzene, kerosene, tetrahydropyran, 1,2,3,4-tetrahydronaphthalene, pentanol and higher homologous alcohols, glycols and their ethers, such as, for example, diethylene glycol dibutyl ether, esters, such as methyl benzoate, ethers, such as ortho-nitrophenyl pentyl ether or nitrophenyl octyl ether, and their mixtures.

22. The supported liquid membrane as claimed in claim 18, wherein the support is a support of inorganic origin, chosen from the group comprising of a silica gel, oxides, such as alumina, zirconia and titanium oxide, or of organic origin, chosen from the group comprising of polystyrene/divinylbenzenes, polyethers, polyacrylamides and poly(glycidyl methacrylate)s, or of organo-inorganic origin, chosen from the group comprising of silica/dextran and hydroxyapatite/agarose composites, and their mixtures.

23. The supported liquid membrane as claimed in claim 19, wherein the support is a support of inorganic origin, chosen from the group comprising of a silica gel, oxides, such as alumina, zirconia and titanium oxide, or of organic origin, chosen from the group comprising of polystyrene/divinylbenzenes, polyethers, polyacrylamides and poly(glycidyl methacrylate)s, or of organo-inorganic origin, chosen from the group comprising of silica/dextran and hydroxyapatite/agarose composites, and their mixtures.

24. The supported liquid membrane as claimed in claim 21, wherein the support is a support of inorganic origin, chosen from the group comprising of a silica gel, oxides, such as alumina, zirconia and titanium oxide, or of organic origin, chosen from the group comprising of polystyrene/divinylbenzenes, polyethers, polyacrylamides and poly(glycidyl methacrylate)s, or of organo-inorganic origin, chosen from the group comprising of silica/dextran and hydroxyapatite/agarose composites, and their mixtures.

25. The supported liquid membrane as claimed in claim 18, wherein the support is a particulate support, the particle size of which varies between 10 nm and 10 mm, preferably between 10 and 50 microns, and the pore diameter of which varies between 10 and 5000 Å, preferably between 100 and 500 Å.

26. The supported liquid membrane as claimed in claim 19, wherein the support is a particulate support, the particle size of which varies between 10 nm and 10 mm, preferably between 10 and 50 microns, and the pore diameter of which varies between 10 and 5000 Å, preferably between 100 and 500 Å.

27. The supported liquid membrane as claimed in claim 21, wherein the support is a particulate support, the particle size of which varies between 10 nm and 10 mm, preferably between 10 and 50 microns, and the pore diameter of which varies between 10 and 5000 Å, preferably between 100 and 500 Å.

28. The supported liquid membrane as claimed in claim 22, wherein the support is a particulate support, the particle size of which varies between 10 nm and 10 mm, preferably between 10 and 50 microns, and the pore diameter of which varies between 10 and 5000 Å, preferably between 100 and 500 Å.

29. The supported liquid membrane as claimed in claim 23, wherein the support is a particulate support, the particle size of which varies between 10 nm and 10 mm, preferably between 10 and 50 microns, and the pore diameter of which varies between 10 and 5000 Å, preferably between 100 and 500 Å.

30. The supported liquid membrane as claimed in claim 24, wherein the support is a particulate support, the particle size of which varies between 10 nm and 10 mm, preferably between 10 and 50 microns, and the pore diameter of which varies between 10 and 5000 Å, preferably between 100 and 500 Å.

31. A support material which is a para-tert-butyl-calix[6Jarene of formula (IIA) or (IIB) 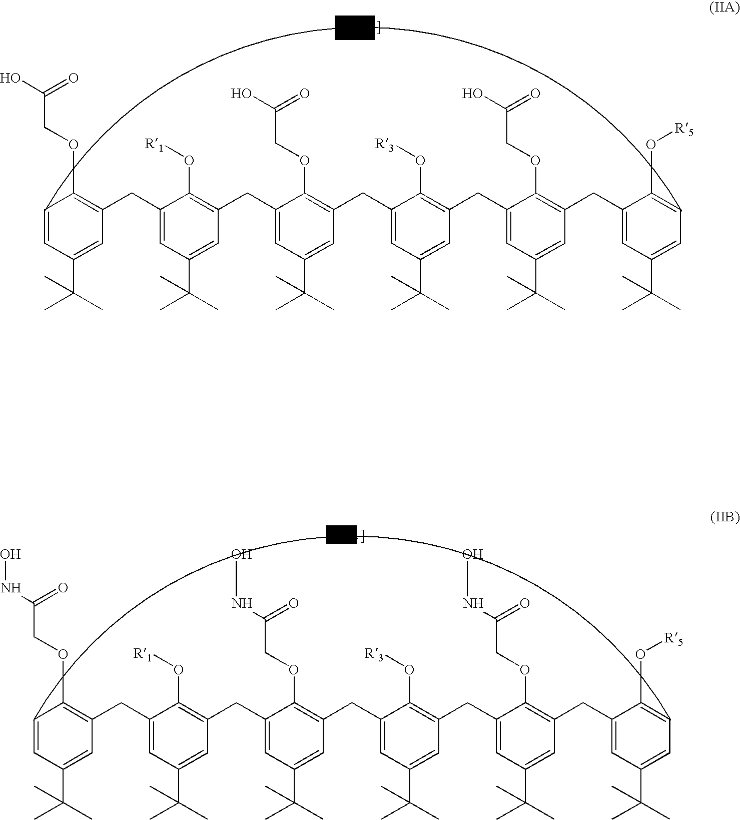
where R′1, R′3 and R′S, which are identical or different, represent, each independently:
(i) a hydrogen or halogen atom,
(ii) an acetyl, amino, phosphate, nitro, sulfate, carboxyl, carboxylic, thiocarboxyl, carbamate or thiocarbamate radical,
(iii) an optionally substituted linear or branched alkyl having 1 to 60, preferably 1 to 30, carbon atoms which optionally exhibits at least one ethylenic or acetylenic unsaturation,
(iv) an optionally substituted cycloalkyl having from 3 to 12 carbon atoms which optionally exhibits at least one ethylenic or acetylenic unsaturation,
(v) an optionally substituted aryl, an optionally substituted naphthyl, an optionally substituted aryl(C₁-C₃₀ alkyl) or an optionally substituted (C₁-C₃₀ alkyl)aryl;
it being possible for the radicals (ii) to (v) to be substituted by halogen atoms, organometallic compounds, alcohol, amine, carboxylic, sulfonic, sulfuric, phosphoric, phosphonic or hydroxamic acid or ester, carbamate, thiocarbamate, ether, thiol, epoxide, thioepoxide, isocyanate or isothiocyanate functional groups or it being possible for a carbon of these radicals to be replaced by a nitrogen, sulfur, phosphorus, oxygen, boron or arsenic heteroatom;
(vi) a polymer chosen from the group comprising of polystyrenes, copolymers of chloro- and/or bromomethylstyrene and of divinylbenzene, polyethers, polyacrylamides, poly(glycidyl methacrylate)s, dextrans and agaroses;
(vii) -SPACER-SUPPORT,
the SPACER being a divalent radical chosen from the group comprising of C₁-C₆₀, preferably C₁-C₃₀, alkylenes, (C₁-C₆ alkyl)arylenes, aryl(C₁-C₆₀ alkylenes) and aryl(C₁-C₆₀ alkyl)aryls, it being possible for the divalent radical to be substituted by halogen atoms, organometallic compounds, alcohol, amine, acid, ester, carbamate, thiocarbamate, ether, thiol, epoxide, thioepoxide, isocyanate or isothiocyanate functional groups or it being possible for a carbon of this divalent radical to be replaced by a nitrogen, sulfur, phosphorus, oxygen, boron or arsenic heteroatom; the SUPPORT being chosen from supports of inorganic or organic or organo-inorganic origin, preferably particulate supports, the particle size of which varies between 10 nm and 10 mm, preferably between 10 and 50 microns, and the pore diameter of which varies between 10 and 5000 Å, preferably between 100 and 500 Å;
provided that at least one of R′1, R′3 or R′S is a (vi) or (vii) group.

32. The support material as claimed in claim 31, wherein the SUPPORT is a support of inorganic origin, chosen from the group comprising of silica gels, oxides, such as alumina, zirconia and titanium oxide, or of organic origin, chosen from the group comprising of polystyrene/divinylbenzenes, polyethers, polyacrylamides and poly(glycidyl methacrylate)s, or of organo-inorganic origin, chosen from the group comprising of silica/dextran or hydroxyapatite/agarose composites.

33. Method for the selective complexing and the analysis of the elements uranium, americium and plutonium or other radioelements in their cationic form, comprising a step of using a supported liquid membrane as claimed in claim 18.

34. Method for removing, from a mixture of at least two constituents chosen from the group comprising of organic, inorganic or organo-inorganic molecules, at least a portion of one of these constituents or for separating said constituents by a chromatographic method, comprising a step of using a support material as claimed in claim 31.
